(19) United States
(12) Patent Application Publication
(10) Pub. No.: US 2024/0182377 A1
(43) Pub. Date: Jun. 6, 2024

(54) PLANT AND METHOD FOR PRODUCING UREA GRANULES

(71) Applicants: thyssenkrupp Fertilizer Technology GmbH, Dortmund (DE); thyssenkrupp AG, Essen (DE)

(72) Inventor: Harald FRANZRAHE, Dortmund (DE)

(73) Assignees: thyssenkrupp Fertilizer Technology GmbH, Dortmund (DE); thyssenkrupp AG, Essen (DE)

(21) Appl. No.: 18/283,322

(22) PCT Filed: Mar. 23, 2022
(86) PCT No.: PCT/EP2022/057707

§ 371 Date: Sep. 21, 2023

(30) Foreign Application Priority Data
Mar. 24, 2021 (DE) 10 2021 202 869.1

(51) Int. Cl. C05C 9/00; B01D 47/06; B01D 53/00

(52) U.S. Cl. CPC C05C 9/005; B01D 47/06; B01D 53/002; B01D 2257/30

(57) ABSTRACT

An installation for the production of granular urea, having at least one urea granulator, at least one dust scrubber, at least one concentrating device, and at least one condensation device, wherein an exhaust gas flow from the urea granulator can be fed to the dust scrubber, wherein the exhaust gas flow is washed in the dust scrubber, wherein at least one outflow from the dust scrubber can be fed to the concentrating device, wherein the outflow can be concentrated in the concentrating device, wherein the vapors created during concentration can be fed, at least in part, to the condensation device, and wherein the vapors are at least partially condensed in the condensation device.

The invention relates to an installation for the production of granular urea, having at least one urea granulator, at least one dust scrubber, at least one concentrating device, and at least one condensation device, wherein an exhaust gas flow from the urea granulator can be fed to the dust scrubber, wherein the exhaust gas flow is washed in the dust scrubber, wherein at least one outflow from the dust scrubber can be fed to the concentrating device, wherein the outflow can be concentrated in the concentrating device, wherein the vapors created during concentration can be fed, at least in part, to the condensation device, and wherein the vapors are at least partially condensed in the condensation device.

In addition, the invention relates to a method for the production of granular urea, wherein an exhaust gas flow from the urea granulator is washed in a dust scrubber, wherein at least one outflow from the dust scrubber is concentrated and fed to the urea granulator, wherein the vapors produced during concentration are condensed at least in part in a condensation device.

The large-scale production of urea almost exclusively involves the use of high-pressure synthesis of ammonia (NH₃) and carbon dioxide (CO₂) at roughly 150 bar and approx. 180 degrees Celsius. The two ingredients are frequently obtained from a neighboring ammonia installation.

Various methods for the production of particulate, urea-containing compounds are known in the prior art. In the past, urea particles were usually produced by means of spray crystallization, wherein a substantially anhydrous urea melt (water content of 0.1 to 0.3% by wt.) is sprayed from the upper part of a spray crystallization tower into a rising flow of air at ambient temperature, and the drops solidify into crystals (prills). The prills which are thereby obtained have relatively small diameters and a low mechanical strength.

Nowadays, urea particles with larger particle diameters and improved mechanical properties are usually produced by granulating a substantially anhydrous urea melt or an aqueous urea solution in a fluid bed. During this granulation process, an aqueous urea solution with a urea concentration of 70-99.9% by wt. in the form of very finely distributed droplets with an average diameter of 20-120 micrometers is added to a fluid bed of urea particles, the temperature being selected such that the water of the solution sprayed onto the urea particles is evaporated and urea is deposited on the particles, so that a granulate with a desired particle size of 2.5 mm, or more, is obtained.

The outgoing air from a fluid bed granulator contains a more or less large fraction of dust. This outgoing air has to be cleaned before it is allowed back into the environment. In most cases, this involves the use of different types of dust scrubbers. In the case of the fluid bed granulation process which is customary today in the production of granular urea, between 2-5% of the product flow can enter the outgoing air as dust. With installation capacities of between 2000 and 4000 tons per day, it is worth integrating the separated dust back into the process. The outflow from the dust scrubbers (approx. 35-50% by wt. urea) is therefore usually concentrated by evaporation (95-99% by wt. urea) before the solution is fed back to the granulation. In the case of pure urea, which is readily water-soluble, this procedure represents the general prior art.

However, the agronomic requirements made of urea fertilizers are changing. There is an increasing need for further nutrients, in order to guarantee an adequate supply of plants. Some of these secondary and micronutrients are not water-soluble, however. This is the case with elementary sulfur, for example. Although water-insoluble substances can usually be included in the urea granulation process without any major problems, these substances are also present in the dust and are separated by the dust scrubbers.

The concentration of urea solutions usually takes place in a vacuum in a bundled tube evaporator. The concentrated solution is once again fed to the granulator. The vapors are then condensed in a bundled tube evaporator. The condensate can be used in the scrubbers to saturate the hot outgoing air from the granulator. If the scrubber solution contains elementary sulfur particles, for example, sulfur can be sublimated from the solid state into the gas phase in the evaporator, due to the high steam pressure. This sublimated sulfur is deposited at each cooler point of the installation and therefore blocks the apparatus in the medium term.

It is relatively difficult to remove sulfur, particularly in a “cold” vapor condenser. The vapors are usually conducted into the tube bundle of a bundled tube condenser. The sulfur thereby reaches the cooling tubes and blocks them. The tubes can only be cleaned mechanically. This necessitates prolonged downtimes and cleaning times.

The object of the present invention is therefore that of specifying an installation and a method for the production of granular urea, in which a clogging of parts of the installation by non-water-soluble substances can be avoided wherever possible.

This object is initially achieved by patent claim 1, in that possible deposits of non-water-soluble substances, in particular sulfur, in the condensation device can be removed from the condensation device during ongoing operation. The non-water-soluble substances in this case can also be removed from the condensation device in such a manner that deposits do not even occur. It is provided in this case that if substances should adhere to the walls of the condensation device, they are washed off or detached from the wall by means of shear force by a flow that is introduced. It is also conceivable for non-water-soluble substances to be melted by a rise in temperature and to become detached from the walls in this way. Non-water-soluble substances in this case should be understood to mean sulfur, in particular, as sulfur is becoming an ever-increasingly important nutrient in the fertilizer industry.

Depending on the desired final concentration (70-99.9% by wt.), the outflow can be concentrated in multiple stages. Each stage in the concentration is then operated at a different pressure. Each concentrating stage is provided with condenser interconnections. Alternatively, a joint condensation stage can be used. Concentration is preferably carried out in the low-pressure range, in other words the operating pressure is lower than the ambient pressure. Higher pressures are likewise possible, however. The installation may then have multiple concentrating devices and multiple outflows as a result.

In a first embodiment of the installation according to the invention, it is provided that the condensation device comprises at least a first condenser and a second condenser, that vapors can flow through the condensers independently of one another, and that during ongoing operation, vapors can selectively flow either through the first condenser or through the second condenser. By using at least two condensers, it is possible for only one condenser to be operational in process terms. If non-water-soluble substances should clog up the tubes or walls of the first condenser, the second condenser can be put into operation. Over the period in which the second condenser is operating, the first condenser can be cleaned. In this case, for example, a heat exchange medium can be conducted through the cooling tubes of the condenser which has been taken out of operation, which medium is at a sufficiently high temperature for the set, non-water-soluble substance to melt and be detached from the wall. The first condenser and/or the second condenser in this case may be a bundled tube condenser.

In a further preferred embodiment of the invention, it is provided that the first condenser or the second condenser can be selectively operated either with cooling water or with steam. If a non-water-soluble substance, for example sulfur, has set on the walls of a condenser, rather than cooling water, steam can be conducted through the cooling tubes. The vapors which continue to be produced can be conducted to the parallel condenser. If steam is conducted through the cooling tubes at a sufficiently high temperature, the non-water-soluble substance melts and becomes detached from the walls of the condenser. The steam should be at a temperature of at least 115 degrees Celsius, preferably 125 degrees Celsius, particularly preferably roughly 135 degrees Celsius. Elementary sulfur has a melting temperature of roughly 115 degrees Celsius. Sulfur can be guaranteed to melt at a temperature of 135 degrees Celsius, in other words with a temperature difference of roughly 20 degrees Celsius in relation to the melting temperature.

For the further configuration of the installation, it is provided in a further embodiment that the first condenser and/or the second condenser is/are designed as a U-tube condenser.

In the case of an alternative or additional embodiment of the invention, it is provided that the condensation device comprises at least one spray condenser. The design of a spray condenser is comparatively simple. It is made up of a tower-like vessel in which cooling water is sprayed.

The high volumes of liquid mean that non-water-soluble substances are prevented from being able to settle on the walls of the spray condenser from the outset.

In order to improve the separation of non-water-soluble substances, in a further embodiment it is provided that the concentrating device is fluidically connected to the condensation device, and that the fluidic connection can be heated. Standard pipeline connections may be used for the fluidic connection. Heating the fluidic connection prevents non-water-soluble substances from cooling down in the fluidic connection to the extent that they can be deposited in the pipelines.

A further embodiment of the invention envisages that the fluidic connection comprises at least one flange connection. The flange connection also has a heatable design, so that non-water-soluble substances are prevented from being deposited in the flange connection.

For further improvement of the separation of sulfur, in particular, it is provided in a further embodiment of the invention that the fluidic connection can be heated in such a manner that the temperature is greater than 115 degrees Celsius, preferably greater than 125 degrees Celsius, particularly preferably 135 degrees Celsius. Elementary sulfur has a melting temperature of roughly 115 degrees Celsius. A temperature of over 115 degrees should therefore be reached as a minimum. A temperature of roughly 135 degrees Celsius is particularly preferable, because a temperature difference of roughly 20 degrees Celsius guarantees the melting of elementary sulfur on the walls of the fluidic connection.

For a more efficient use of the installation, it is provided in a further preferred embodiment of the invention that the condensation device comprises a pump through which the spray water of the spray condenser can be circulated, and that the condensation device comprises a heat exchanger through which the spray water of the spray condenser can be cooled. Hence, the required spray water or cooling water can be circulated and reused. The heat exchanger may be a plate heat exchanger. The flow of water leaving the spray condenser contains both the sulfur, for example, and the condensed vapors. It is also conceivable for a flow control to be provided, by means of which the circulating water flow can be kept constant. The condensate which is produced with a fraction of sulfur, for example, is fed to the scrubbing system, where it is used as make-up liquid.

The aforementioned object is, moreover, achieved by a method for the production of granular urea, wherein an exhaust gas flow from a urea granulator is washed in a dust scrubber, wherein at least one outflow from the dust scrubber is concentrated and fed to the urea granulator, wherein the vapors produced during concentration are condensed at least in part in a condensation device, characterized in that possible deposits of non-water-soluble substances, in particular sulfur, in the condensation device are removed from the condensation device during ongoing operation.

The method can be carried out using an aforementioned installation. The comments made in relation to the installation according to the invention also apply in the same way to the method according to the invention.

A first embodiment of the method according to the invention provides that the condensation device comprises at least a first condenser and a second condenser, that vapors can flow through the condensers independently of one another, and that it is possible to switch from the first condenser to the second condenser, and vice versa, during ongoing operation.

A further embodiment of the method provides that the first condenser or the second condenser can be selectively operated either with cooling water or with steam. The steam is preferably at a temperature that lies above the melting temperature of the non-water-soluble substances located in the mass flow. In this way, the non-water-soluble substances which have been deposited on the walls of the first or second condenser are melted and transported out of the condenser. Operating the condenser with cooling water or steam means that either cooling water or steam is conducted through the cooling tubes of the condenser. In this way, either the cooling water absorbs the heat from the mass flow, which is condensed, or, however, the set, non-water-soluble substances absorb the heat from the steam and melt. There is no substance exchange between the heat exchange medium, in other words the steam or the cooling water, and the mass flow or the non-water-soluble substances in the condenser.

A preferred embodiment of the method according to the invention provides that the condensation device comprises at least one spray condenser and that the vapors from the concentrating device are fed at least in part via a heatable fluidic connection to the condensation device.

In order to be able to separate sulfur, in particular, with a preferred embodiment of the method according to the invention, it is provided that the heatable fluidic connection is brought to a temperature greater than 115 degrees Celsius, preferably greater than 125 degrees Celsius, particularly preferably 135 degrees Celsius.

Specifically, there is a plurality of possible ways of configuring and developing the installation according to the invention and the method according to the invention. For this purpose, reference is made both to the patent claims subordinate to patent claims 1 and 10, and to the following description of preferred exemplary embodiments, in conjunction with the drawing. In the drawing

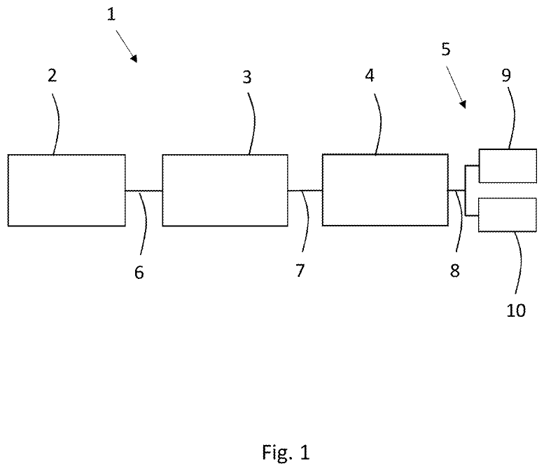

FIG. 1 shows a schematic representation of a part of a urea granulation process with sulfur recovery by means of a redundant heat exchanger, and

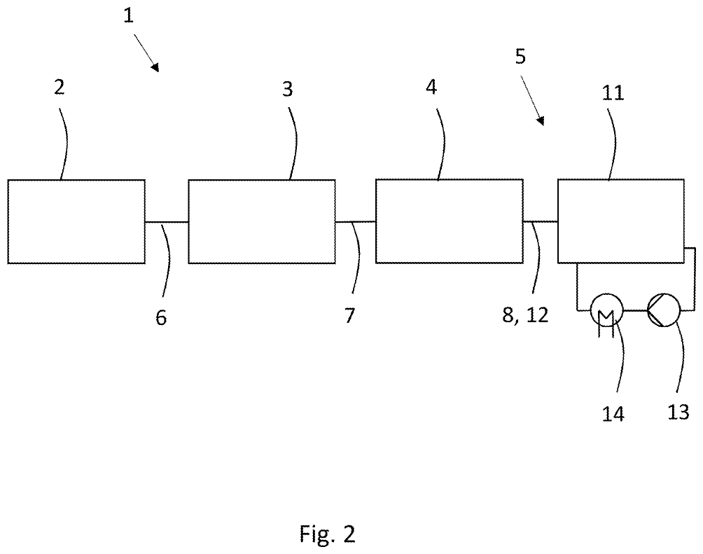

FIG. 2 shows a schematic representation of a part of a urea granulation process with sulfur recovery by means of spray condensation.

FIG. 1 shows a schematic representation of an installation 1 for producing granular urea by means of a urea granulator 2. The installation comprises, among other things, a dust scrubber 3, a concentrating device 4, and a condensation device 5. An exhaust gas flow 6 coming from the urea granulator 2 contains dust and is therefore fed to the dust scrubber 3. Particularly when granular urea is being produced, the outgoing air which leaves the urea granulator 2 in the form of a fluid bed granulator must be cleaned before it can go back into the environment. The increased requirements made of fertilizers mean that further nutrients are needed, which are easily granulated into the product, but which under certain circumstances can set in the installation as a solid. One of these nutrients is sulfur.

The outflow 7 leaving the dust scrubber 3 is fed to the concentrating device 4. By means of evaporation, the urea solution is vacuum-concentrated in the concentrating device 4 in a bundled tube evaporator which is not depicted here. The concentrated urea solution can be fed back to the urea granulator 2. The vapors 8 which leave the concentrating device 4 are condensed in the condensation device 5. Since sulfur has a relatively high steam pressure, for example, solid sulfur is partially sublimated in the concentrating device 4 into the gas phase, and is likewise contained in the vapors 8. The condensate can be used again to saturate the hot outgoing air from the urea granulator 2 in the dust scrubber 3.

In the case of the exemplary embodiment shown in FIG. 1, the condensation device 5 comprises a first condenser 9 and a second condenser 10. Further condensers are also conceivable, however. The condensers 9, 10 are configured as U-tube condensers. The first condenser 9 and the second condenser 10 are connected together in such a manner that the first condenser 9 can be taken out of service, wherein at the same time the second condenser 10 comes into service. In other words, it is possible to switch from the first condenser 9 to the second condenser 10 during ongoing operation. The hot vapors 8 are condensed in the first condenser 9, for example.

Apart from urea, the vapors 8 also contain further nutrients such as sulfur, for example. This gaseous sulfur condenses in the first condenser 9 and sets on the cold points, in other words on the walls of the first condenser 9. When the condenser 9 is clogged up to such an extent that operation of the installation 1 is possibly jeopardized, it is possible to switch to the second condenser 10, which has no sulfur on its walls, during ongoing operation. The first condenser 9 and the second condenser 10 are designed in such a manner that they can be operated as a heat exchange medium, both using cooling water and using steam. When the first condenser 9 is taken out of service, it can be “regenerated” by conducting steam at roughly 135 degrees Celsius through the cooling tubes of the first condenser 9 instead of cooling water. The steam heats the walls of the first condenser 9, to the extent that the sulfur which has set on the walls melts and can be removed from the first condenser 9. In this way, the first condenser 9 is once again ready for use. Consequently, it is possible to switch over to the first condenser 9 when the second condenser 10 has become clogged with non-water-soluble substances, to the extent that removal of these substances becomes necessary.

FIG. 2 shows a similar design of an installation 1 for the production of granular urea, as already described in FIG. 1. Unlike in FIG. 1, the condensation device 5 does not comprise a first and second condenser 9, 10, but a spray condenser 11. The spray condenser 11 is a relatively simple piece of apparatus. It is made up of a tower-like vessel in which cooling water is sprayed. The large volumes of liquid mean that elementary sulfur, which condenses from the gas phase, is prevented from the outset from settling in the spray condenser 11 on the walls of the spray condenser 11.

The spray condenser 11, or else the condensation device 5, is connected to the concentrating device 4 by means of a fluidic connection 12, in this case via pipe and flange connections. The fluidic connection 12 has a heated design, so that sulfur cannot condense in the fluidic connection 12 and clog up the pipe and flange connections. In the spray condenser 12, this is prevented by the sprayed-in water. The temperature of the fluidic connection 12 is set at roughly 135 degrees Celsius, so that a temperature difference of roughly 20 degrees Celsius in respect of the melting temperature of sulfur prevails.

The required spray water is circulated using a pump 13 and cooled by means of a heat exchanger 14 in the form of a plate heat exchanger. The flow of water leaving the spray condenser 12 contains both the separated sulfur and the condensed vapors 8. The circulating water flow of the spray condenser can be kept constant via a flow control which is not depicted here. The condensate water which is produced with a fraction of sulfur can be fed to the dust scrubber 3, where it is used as make-up liquid.

### LIST OF REFERENCE SIGNS

(1) Installation
(2) Urea granulator
(3) Dust scrubber
(4) Concentrating device
(5) Condensation device
(6) Exhaust gas flow
(7) Outflow
(8) Vapors
(9) First condenser
(10) Second condenser
(11) Spray condenser
(12) Fluidic connection
(13) Pump
(14) Heat exchanger

1-14. (canceled)

15. An installation for the production of granular urea, comprising:
an urea granulator;
a dust scrubber;
a concentrating device; and
a condensation device, wherein an exhaust gas flow from the urea granulator is arranged to be fed to the dust scrubber, wherein the exhaust gas flow is configured to be washed in the dust scrubber, wherein at least one outflow from the dust scrubber is configured to be fed to the concentrating device, wherein the outflow is configured to be concentrated in the concentrating device, wherein vapors created during concentration are configured to be fed, at least in part, to the condensation device, and wherein the vapors are at least partially configured to be condensed in the condensation device, and possible deposits of non-water-soluble substances in the condensation device are avoided during operation or removed from the condensation device during operation.

16. The installation of claim 15, wherein the condensation device comprises at least a first condenser and a second condenser, configured such that vapors are able to flow through the condensers independently of one another, and that during operation, vapors can selectively flow either through the first condenser or through the second condenser.

17. The installation of claim 16, wherein the first condenser or the second condenser are selectively operated either with cooling water or with steam.

18. The installation of claim 16, wherein the first condenser and/or the second condenser is/are designed as a U-tube condenser.

19. The installation of claim 15, wherein the condensation device comprises at least one spray condenser.

20. The installation of claim 15, wherein the concentrating device is fluidically connected to the condensation device, and that the fluidic connection is configured to be heated.

21. The installation of claim 20, wherein the fluidic connection comprises at least one flange connection.

22. The installation of claim 20, wherein the fluidic connection is configured to be heated in such that the temperature is greater than 115 degrees Celsius.

23. The installation of claim 19, wherein the condensation device comprises a pump through which the spray water of the spray condenser is configured to be circulated, and that the condensation device comprises a heat exchanger through which the spray water of the spray condenser is configured to be cooled.

24. A method for the production of granular urea, comprising:
washing an exhaust gas flow from a urea granulator in a dust scrubber, wherein at least one outflow from the dust scrubber is concentrated and fed to the urea granulator; and
condensing vapors produced during concentration at least in part in a condensation device; and
removing deposits of non-water-soluble substances in the condensation device from the condensation device during operation.

25. The method of claim 24, wherein the condensation device comprises at least a first condenser and a second condenser, wherein vapors can flow through the condensers independently of one another, and that it is possible to switch from the first condenser to the second condenser, and vice versa, during ongoing operation.

26. The method of claim 25, wherein the first condenser or the second condenser can be selectively operated either with cooling water or with steam.

27. The method of claim 24, wherein the condensation device comprises at least one spray condenser and that the vapors from the concentrating device are fed at least in part via a heatable fluidic connection to the condensation device.

28. The method of claim 27, wherein the heatable fluidic connection is brought to a temperature greater than 115 degrees Celsius.
